(19) United States
(12) Patent Application Publication
(10) Pub. No.: US 2011/0295053 A1
(43) Pub. Date: Dec. 1, 2011

(54) Implantable Inner Ear Drive System

(75) Inventor: Geoffrey R. Ball, Axams (AT)

(73) Assignee: VIBRANT MED-EL HEARING TECHNOLOGY GMBH, Innsbruck (AT)

(21) Appl. No.: 13/114,264

(22) Filed: May 24, 2011

Related U.S. Application Data
(60) Provisional application No. 61/348,973, filed on May 27, 2010.

(51) Int. Cl. A61F 11/04

(52) U.S. Cl. 600/25

(57) ABSTRACT

An acoustic drive device for an implantable hearing prosthesis is described. A cantilevered positioning stalk has a base end fixedly coupled to an implantable signal processor, an elongated center beam supported by the base end, and an unsupported free end of the positioning stalk. An acoustic drive unit is located at the free end of the positioning stalk and adapted to convert an electrical stimulation signal from the signal processor into an acoustic mechanical stimulation signal directed to an outer surface of a patient cochlea.

This application claims priority from U.S. Provisional patent application 61/348,973, filed May 27, 2010; which is incorporated herein by reference.

### FIELD OF THE INVENTION

The present invention relates to medical implants, and more specifically to a novel acoustic drive unit for implantable auditory prosthetic systems.

### BACKGROUND ART

A normal ear transmits sounds as shown in FIG. 1 through the outer ear 101 to the tympanic membrane (eardrum) 102, which moves the ossicles of the middle ear 103 (malleus, incus, and stapes) that vibrate the oval window 106 and round window 107 membranes of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolar where the spiral ganglion cells of the cochlear nerve 105 reside. In response to received sounds transmitted by the middle ear 103, the fluid-filled cochlea 104 functions as a transducer to generate electric pulses which are transmitted to the cochlear nerve 105, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is related to operation of the middle ear 103, a conventional hearing aid or middle ear implant may be used to provide acoustic-mechanical stimulation to the auditory system in the form of amplified sound. Or when the impairment is associated with the cochlea 104, a cochlear implant with an implanted stimulation electrode can electrically stimulate auditory nerve tissue with small currents delivered by multiple electrode contacts distributed along the electrode.

Traditional middle ear implants employ electromagnetic transducers to convert sounds into mechanical vibration of the middle ear 103. A coil winding is held stationary by attachment to a non-vibrating structure within the middle ear 103 and microphone signal current is delivered to the coil winding to generate an electromagnetic field. A magnet is attached to an ossicle within the middle ear 103 so that the magnetic field of the magnet interacts with the magnetic field of the coil. The magnet vibrates in response to the interaction of the magnetic fields, causing vibration of the bones of the middle ear 103. See U.S. Pat. No. 6,190,305, which is incorporated herein by reference.

### SUMMARY OF THE INVENTION

Embodiments of the present invention are directed to an acoustic drive device for an implantable hearing prosthesis. A cantilevered positioning stalk has a base end fixedly coupled to an implantable signal processor, an elongated center beam supported by the base end, and an unsupported free end of the positioning stalk. An acoustic drive unit is located at the free end of the positioning stalk and adapted to convert an electrical stimulation signal from the signal processor into an acoustic mechanical stimulation signal directed to an outer surface of a patient cochlea such as the round window membrane or oval window membrane.

The center beam may be deformable to position the acoustic drive unit relative to the outer surface of the patient cochlea. The center beam also may include a locking mechanism for fixing the acoustic drive unit relative to the positioning stalk. The center beam may include silicone tubing and/or a center support rod.

The acoustic drive unit may include an electromagnetic transducer. The electromagnetic transducer may include an output diaphragm for acoustically or mechanically driving the acoustic mechanical stimulation signal to the outer surface of the patient cochlea. The electromagnetic transducer and the positioning stalk may have outer diameters that are about the same.

There may be a magnetic drive lens having a coupling end in sliding magnetic engagement with the electromagnetic transducer and a drive end in direct contact with the outer surface of the patient cochlea. The drive end may include a planar or curved drive surface in direct contact with the outer surface of the patient cochlea. And there may be a coupling spring resiliently connecting the coupling end to the electromagnetic transducer.

Embodiments of the present invention also include an acoustic drive device for an implantable hearing prosthesis which includes an acoustic drive unit adapted to convert an electrical stimulation signal into an acoustic mechanical stimulation signal directed to an outer surface of a patient cochlea, and located at the free end of a positioning member, wherein the acoustic drive unit and the positioning member have outer diameters that are about the same. The outer surface may include the round window membrane or the oval window membrane of the cochlea. The acoustic drive unit may include an electromagnetic transducer, which may have an output diaphragm for acoustically or mechanically driving the acoustic mechanical stimulation signal to the outer surface of the patient cochlea.

There may be a magnetic drive lens having a coupling end in sliding magnetic engagement with the electromagnetic transducer and a drive end in direct contact with the outer surface of the patient cochlea. The drive end may include a planar or curved drive surface in direct contact with the outer surface of the patient cochlea. A coupling spring may resiliently connect the coupling end to the electromagnetic transducer.

### BRIEF DESCRIPTION OF THE DRAWINGS

FIG. 1 shows various anatomical structures in a normal human ear.

FIG. 2 shows a specific embodiment of the present invention having a positioning stalk that places a small acoustic drive unit adjacent to the outer surface of a patient cochlea.

FIG. 3 A-B shows greater structural details of one specific embodiment of an acoustic drive unit having a curved drive surface end.

FIG. 4 shows another embodiment of an acoustic drive unit having a drive diaphragm.

FIG. 5 shows a magnetic drive lens having a planar drive surface.

FIG. 6 shows an acoustic drive unit having a magnetic drive lens arrangement according to one embodiment of the present invention.

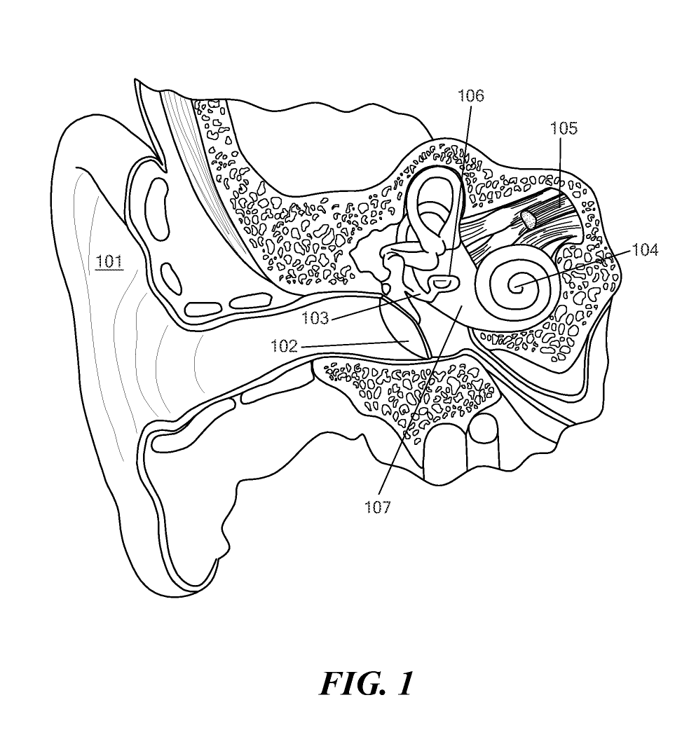

### DETAILED DESCRIPTION OF SPECIFIC EMBODIMENTS

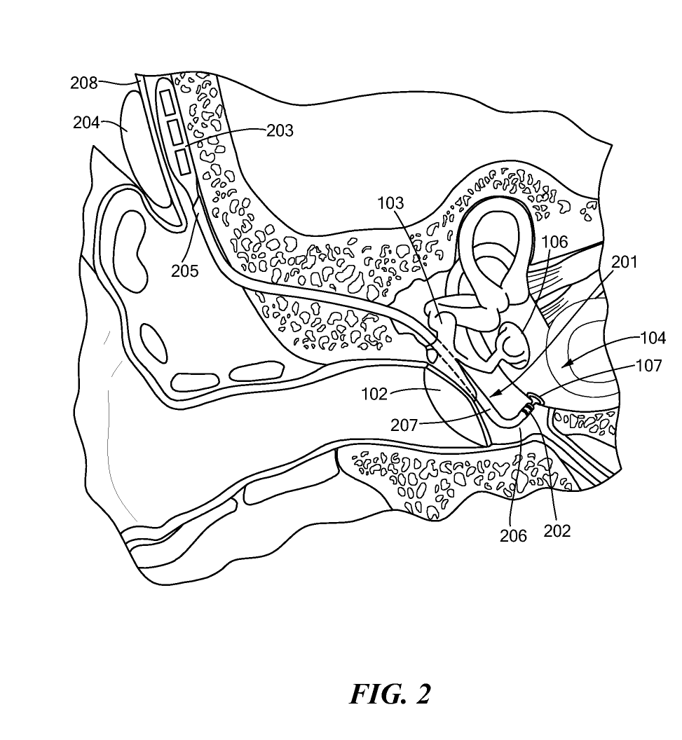

Various embodiments of the present invention are directed to an acoustic drive device having a having a positioning stalk that places a small acoustic drive unit adjacent to the outer surface of a patient cochlea. FIG. 2 shows a specific embodiment having a cantilevered positioning stalk 201 that has a fixed base end 205, an elongated center beam 207 and an unsupported free end 206. The base end 205 is fixedly coupled to an implant signal processor 203 that receives an implant communications signal through the skin 208 of the patient from an external signal processor 204. The elongated center beam 207 is supported in fixed position by the fixed base end 205. An acoustic drive unit 202 is located at the free end 206 of the positioning stalk 201. The acoustic drive unit 202 is adapted to convert an electrical stimulation signal from the implant signal processor 203 into an acoustic mechanical stimulation signal which is acoustically or mechanically directed to an adjacent outer surface of a patient cochlea 104 such as the round window membrane 107 or oval window membrane 106.

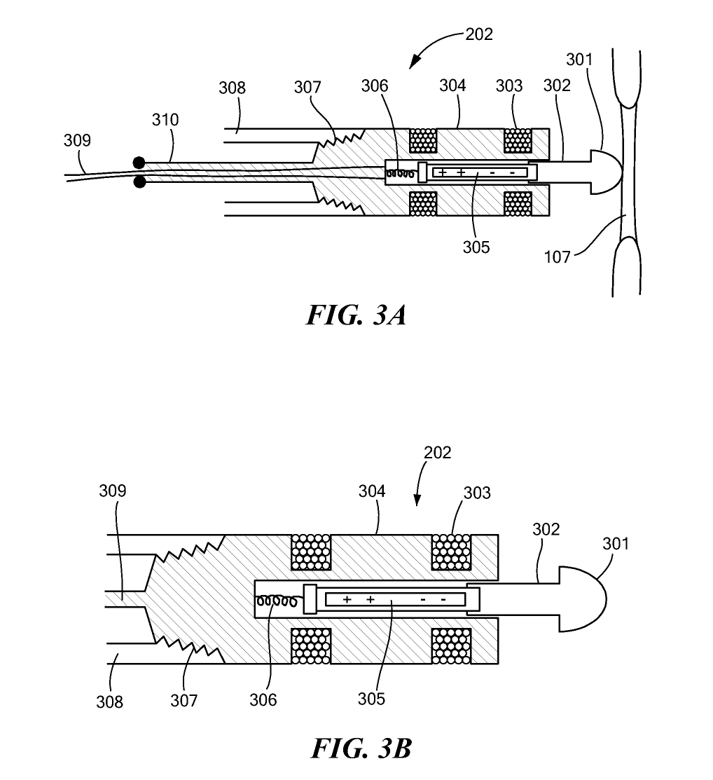

FIG. 3 A-B shows greater structural details of one specific embodiment of an acoustic drive unit 202 about 1-2 mm in diameter and having a titanium window coupler with a curved end drive surface 301 typically about 0.5 to 1.5 mm in diameter which is adapted to directly mechanically engage the outer surface of the patient cochlea 104 such as the round window membrane 107 or the oval window membrane 106. In this embodiment, the acoustic drive unit 202 is an electromagnetic transducer design having a pair of electric drive coils 303 on either side of a center bobbin 304 made of ferromagnetic material. Together the drive coils 303 and center bobbin 304 form a cylindrical magnetic driver surrounding a central axial drive rod 305 formed of a magnetic rod which is coupled at a drive end 302 to the drive surface 301 and at the other end to a coupling spring 306 that resiliently biases the drive rod 305 into a correct position with regards to both the magnetic driver elements and the outer surface of the cochlea. In FIG. 3A, the drive rod 305 is a solid magnetic rod, while FIG. 3B shows another embodiment where the drive rod 305 is a titanium cylinder that hermetically encloses an inner rod magnet.

FIG. 3 A-B also shows interior structural details of the positioning stalk 201 which is formed of hollow silicone tubing 308 about 1-2 mm in outside diameter and enclosing a center support rod 309 made of deformable material such as bendable titanium. The deformable bendability of the support rod 309 allows the acoustic drive unit 202 to be easily positioned during surgical installation to correctly engage the outer surface of the cochlea. A threaded locking end 307 couples the drive unit 202 to the silicone tubing 308 end of the positioning stalk 201. Drive wires 310 within the silicone tubing 308 communicate the electrical stimulation signal from the signal processor 203 to acoustic drive unit 202.

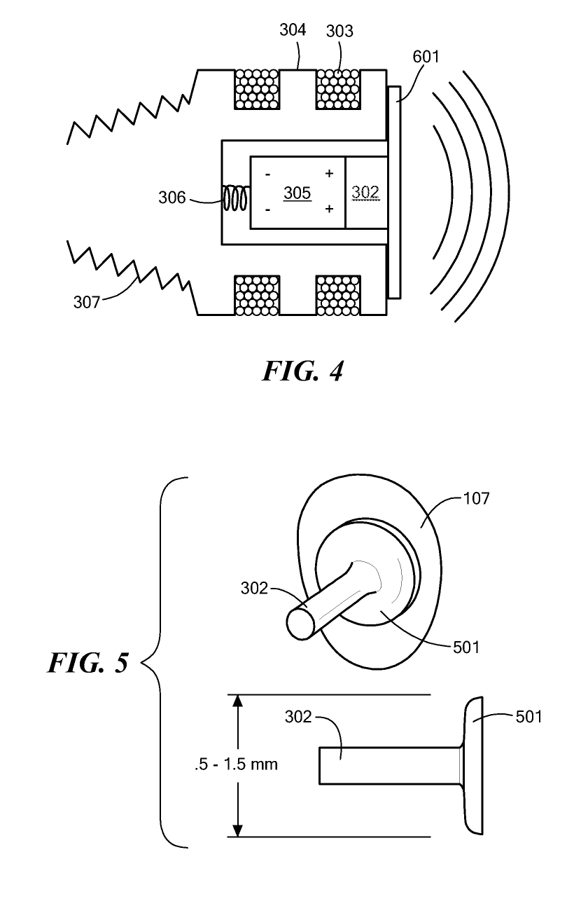

FIG. 4 shows another embodiment of an acoustic drive unit where the drive end 302 of the drive rod 305 is coupled to an output drive diaphragm 601 that is positioned near the outer surface of the patient cochlea 104 rather than in direct mechanical engagement so as to acoustically drive the outer surface with an acoustic stimulation signal based on the electric stimulation signal from the signal processor 203. In other embodiments, the output drive diaphragm 601 may be adapted to directly engage the outer surface of the cochlea 104 to directly mechanically stimulate it.

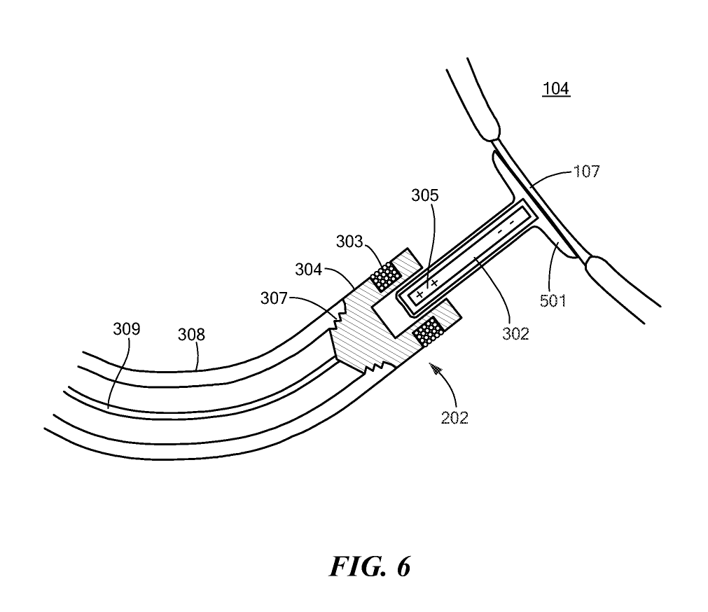

FIG. 5 shows a magnetic drive lens having magnetic drive lens 501 with a planar drive surface which is typically about 0.5-1.5 mm in diameter made of an appropriate bioinert material such as silicone or titanium. FIG. 6 shows an acoustic drive unit having such a magnetic drive lens arrangement. The planar drive surface of the magnetic drive lens 501 is well suited to be tacked onto the outer surface of one of the window membranes of the patient cochlea to directly engage it with a mechanical stimulation signal. The planar drive surface will easily adhere to the soft tissue of the membrane surface, for example, by capillary pressure. The drive end 302 is easily separable from and operates in slidable engagement with the acoustic drive unit 202. Thus during surgery, the surgeon can conveniently install the magnetic lens 501 into position on the outer surface of the window membrane, and then bend the positioning stalk 201 to position the acoustic drive unit 202 into an operating position around the drive end 302 of the magnetic lens 501.

Although various exemplary embodiments of the invention have been disclosed, it should be apparent to those skilled in the art that various changes and modifications can be made which will achieve some of the advantages of the invention without departing from the true scope of the invention.

What is claimed is:

1. An acoustic drive device for an implantable hearing prosthesis comprising:
a cantilevered positioning stalk having:
i. a base end fixedly coupled to an implantable signal processor,
ii. an elongated center beam supported by the base end, and
iii. an unsupported free end of the positioning stalk; and
an acoustic drive unit located at the free end of the positioning stalk and adapted to convert an electrical stimulation signal from the signal processor into an acoustic mechanical stimulation signal directed to an outer surface of a patient cochlea.

2. An acoustic drive device according to claim 1, wherein the center beam is deformable to position the acoustic drive unit relative to the outer surface of the patient cochlea.

3. An acoustic drive device according to claim 1, wherein the center beam includes a locking mechanism for fixing the acoustic drive unit to the positioning stalk.

4. An acoustic drive device according to claim 1, wherein the center beam includes silicone tubing.

5. An acoustic drive device according to claim 1, wherein the center beam includes a center support rod.

6. An acoustic drive device according to claim 1, wherein the outer surface includes the round window membrane of the cochlea.

7. An acoustic drive device according to claim 1, wherein the outer surface includes the oval window membrane of the cochlea.

8. An acoustic drive device according to claim 1, wherein the acoustic drive unit includes an electromagnetic transducer.

9. An acoustic drive device according to claim 8, wherein the electromagnetic transducer includes an output diaphragm for driving the acoustic mechanical stimulation signal to the outer surface of the patient cochlea.

10. An acoustic drive device according to claim 9, wherein the output diaphragm acoustically drives the acoustic mechanical stimulation signal.

11. An acoustic drive device according to claim 9, wherein the output diaphragm mechanically drives the acoustic mechanical stimulation signal.

12. An acoustic drive device according to claim 8, wherein the electromagnetic transducer and the positioning stalk have outer diameters that are about the same.

13. An acoustic drive device according to claim 8, further comprising:
a magnetic drive lens having a coupling end in sliding magnetic engagement with the electromagnetic transducer and a drive end in direct contact with the outer surface of the patient cochlea.

14. An acoustic drive device according to claim 13, wherein the drive end includes a planar drive surface in direct contact with the outer surface of the patient cochlea.

15. An acoustic drive device according to claim 13, wherein the drive end includes a curved drive surface in direct contact with the outer surface of the patient cochlea.

16. An acoustic drive device according to claim 13, further comprising:
a coupling spring resiliently connecting the coupling end to the electromagnetic transducer.

17. An acoustic drive device for an implantable hearing prosthesis comprising:
an acoustic drive unit adapted to convert an electrical stimulation signal into an acoustic mechanical stimulation signal directed to an outer surface of a patient cochlea, and located at the free end of a positioning member;
wherein the acoustic drive unit and the positioning member have outer diameters that are about the same.

18. An acoustic drive device according to claim 17, wherein the outer surface includes the round window membrane of the cochlea.

19. An acoustic drive device according to claim 17, wherein the outer surface includes the oval window membrane of the cochlea.

20. An acoustic drive device according to claim 17, wherein the acoustic drive unit includes an electromagnetic transducer.

21. An acoustic drive device according to claim 20, wherein the electromagnetic transducer includes an output diaphragm for driving the acoustic mechanical stimulation signal to the outer surface of the patient cochlea.

22. An acoustic drive device according to claim 21, wherein the output diaphragm acoustically drives the acoustic mechanical stimulation signal.

23. An acoustic drive device according to claim 21, wherein the output diaphragm mechanically drives the acoustic mechanical stimulation signal.

24. An acoustic drive device according to claim 20, further comprising:
a magnetic drive lens having a coupling end in sliding magnetic engagement with the electromagnetic transducer and a drive end in direct contact with the outer surface of the patient cochlea.

25. An acoustic drive device according to claim 24, wherein the drive end includes a planar drive surface in direct contact with the outer surface of the patient cochlea.

26. An acoustic drive device according to claim 24, wherein the drive end includes a curved drive surface in direct contact with the outer surface of the patient cochlea.

27. An acoustic drive device according to claim 20, further comprising:
a coupling spring resiliently connecting the coupling end to the electromagnetic transducer.
